(19) United States
(12) Patent Application Publication
(10) Pub. No.: US 2019/0282540 A1
(43) Pub. Date: Sep. 19, 2019

(54) TARGETED DRUG DELIVERY

(71) Applicant: NOXOPHARM LIMITED, Turramurra, New South Wales (AU)

(72) Inventor: Graham KELLY, Turramurra (AU)

(21) Appl. No.: 16/091,699

(22) PCT Filed: Apr. 6, 2017
(86) PCT No.: PCT/AU2017/050300

§ 371 Date: Oct. 5, 2018

Related U.S. Application Data
(60) Provisional application No. 62/318,946, filed on Apr. 6, 2016.

(30) Foreign Application Priority Data
Jul. 28, 2016 (AU) PCT/AU2016/050674
Nov. 22, 2016 (AU) 2016904778

(51) Int. Cl. A61K 31/353; A61P 35/00; A61K 31/12

(52) U.S. Cl. CPC A61K 31/353; A61K 31/12; A61P 35/00

(57) ABSTRACT

A method for providing isoflavonoid compounds to the central nervous system comprising the step of rectally administering the compound to an individual in need thereof. This method is useful in the treatment of brain cancers, including glioblastoma multiforme.

### FIELD OF THE INVENTION

This invention relates to isoflavonoids, to formulations for rectal, urethral or vaginal delivery of therapeutic compounds, to pharmacokinetics, and to the delivery of compounds to the central nervous system, especially the brain parenchyma.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Plant-derived phenolic isoflavonoids have been the subject of considerable scientific research since the late-1980s. Many of these compounds have auxin or hormonal functions in plants and also display biological activities in human tissues. One of the most extensively studied plant isoflavones is genistein, remarkable for its pleiotropic actions across carcinogenesis, inflammation, cardiovascular function, and insulin resistance.

The anti-cancer activities of genistein appear to stem in part from its ability to block the phosphorylation of protein tyrosine kinases, resulting in mitotic arrest, terminal differentiation, and apoptosis of human cancer cells (Lambert et al, 2005; Williamson and Manach, 2005). Genistein also is anti-angiogenic (Piao et al, 2006). The anti-cancer effects of genistein also extend to epigenetic modifications of cancer cells through modulation of DNA methylation, miRNA-mediated regulation and histone modifications (Adjakly et al, 2015) and to inhibition of proteasome activity (Kazi et al, 2003).

Importantly, isoflavonoids have been found to be useful as cytotoxic agents, and as sensitising agents for sensitising cancer cells to cytotoxic signals from chemical or radiation insult. Some have also been shown to reverse chemo-resistance.

Despite these potentially valuable therapeutic opportunities of genistein in particular and a wide range of other related plant isoflavonoids in general, those opportunities have failed to date to be translated into the clinic. There are a number of reasons for this. One is that there is a question as to their susceptibility to various degrees to Phase 1 and Phase 2 metabolic processes with resulting decrease in potency and bio-availability, although the extent to which these process influence therapeutic potential has not been completely understood.

Some have attempted to address these deficiencies through the synthesis of analogues of the naturally-occurring isoflavonoids, hopefully by creating new chemical entities with greater biological potency and/or being less susceptible to metabolic processes.

Idronoxil is an analogue of genistein. Idronoxil (phenoxodiol; dehydroequol; Haginin E (2H-1-Benzopyran-7-0,1,3-(4-hydroxyphenyl) is about 10× more potent as an anti-cancer agent compared to genistein, inducing cytostasis and cytotoxicity in a wide range of cancer cell types. Its biological effects include inducing apoptosis, cell cycle arrest, inhibition of angiogenesis, immune modulation and neuro-protection.

Idronoxil has proved to have better drug-like qualities compared to its parent isoflavone compound, genistein, particularly in having greater in vitro anti-cancer activity and in not being particularly susceptible to Phase 1 metabolic processes (Brown et al, 2008). However, idronoxil, in common with members of the isoflavone family, is likely susceptible to Phase 2 metabolic processes, and it is this phenomenon that is believed to account for the lack of meaningful clinical efficacy observed with this family of compounds to some extent.

Isoflavonoid molecules are highly insoluble in water. In common with other water-insoluble xenobiotics as well as water-insoluble internal hormones (steroidal hormones, thyroxine) and bile acids, the body seeks to convert these compounds into a water-soluble form that is excretable via the kidneys (Guy et al, 2008; Zhang et al, 2003). Excretion can occur via the bile, but the rate of biliary excretion is slow compared to urinary excretion, leading the body to seek to convert as much of the xenobiotic into a water-soluble form that is possible.

Compounds such as idronoxil with an underlying phenolic structure share this feature with other phenolic drugs (eg. propofol, paracetamol, naloxone).

One mode of detoxification may involve a family of UDP-glucuronyl transferase enzymes that attach the xenobiotic to the sugar, glucuronic acid, to produce a water-soluble glucuronide conjugate. A secondary, less common detoxification process involves sulfotransferase enzyme activity that yields a water-soluble sulfated conjugate.

These two families of detoxifying enzymes are located principally in the liver and the gut mucosa (King et al, 2000; Guillemette, 2003; Maruo et al, 2005; Wu et al, 2011).

Orally administered idronoxil is completely converted into water-soluble conjugates as a combined effect of transferase activity in the gut mucosa and first-pass liver metabolism; intravenously administered idronoxil also is completely conjugated as a result of second-pass liver metabolism, with a low level of unconjugated drug being drug retained within the cyclodextrin carrier (Howes et al, 2011).

This process of Phase 2 metabolism has a significant impact on the bio-availability of isoflavonoid drugs to target tissue, in particular brain and spinal cord, and in particular because of the blood-brain barrier.

The central nervous system is protected by an effective chemical barrier known as the blood-brain barrier. The barrier is the result in large part of tighter than normal junctions between endothelial cells lining the capillaries within the brain. As a result, only a limited number of compounds (water, gases and certain small molecular weight compounds) are able to diffuse passively from the bloodstream into the brain. Everything else is subject to active transport, requiring dedicated transporters. As a result, xenobiotics in very large part are excluded from the central nervous system because the body lacks a dedicated transporter.

Endogenous phenolic compounds such as steroid hormones are able to cross the blood brain barrier because of dedicated transporters. For example, the organic anion-transporting polypeptide (Oatp) 1c1 is thought to be responsible for enabling the influx of steroid hormones including estradiol-17b-glucuronide across the blood-brain barrier (Westholm et al, 2009).

This transporter availability appears not to be available for the great majority of exogenous phenolics presenting to the central nervous system as Phase 2 metabolites, including isoflavonoids.

In situ studies show that naturally occurring phenolics such as flavonoids are able to cross the blood-brain barrier (Youdim et al, 2004), but these studies were conducted with free compounds and not their Phase 2 metabolites, failing to take into account the in vivo situation.

Where in vivo studies have been done looking at brain levels of the dietary isoflavone, genistein, following dietary challenge, the level of penetration into the brain is very low with a brain-to-blood ratio of 0.04 (Tsai 2005).

Specifically in the case of idronoxil, in a study where [¹⁴C] idronoxil was given orally at a target dose of 20 mg/kg, low levels of isotope (below the limit of reliable measurement) were recorded in the brain and spinal cord suggesting that idronoxil is unable to cross the intact blood-brain barrier (Brown et al. 2008).

Most of the technologies available for overcoming the blood-brain barrier are highly invasive, involving surgical implantation of devices or compositions into brain tissue, often at the site of removal of a neoplastic lesion, or involving injection into the spinal cord CSF.

There is a need for new methods for the delivery of isoflavonoids to the central nervous system, especially to brain parenchyma and related tissue.

There is a need for new methods for the delivery of idronoxil to the central nervous system, especially to brain parenchyma and related tissue.

### SUMMARY OF THE INVENTION

The invention seeks to address one or more of the above mentioned needs and in one embodiment provides a method for providing a compound of general formula (I)

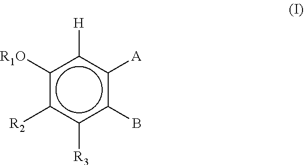

wherein

R₁ is H, C₁₋₁₀ alkyl, aryl, arylalkyl or R_{A}CO where R_{A} is C₁₋₁₀ alkyl or an amino acid;

R₂ is H, OH or OR₆;

R₃ is H, OH, or R_{B} where R_{B} is an amino acid or COR_{A} where R_{A} is as previously defined;

A and B together with the atoms between them form a six membered ring selected from the group

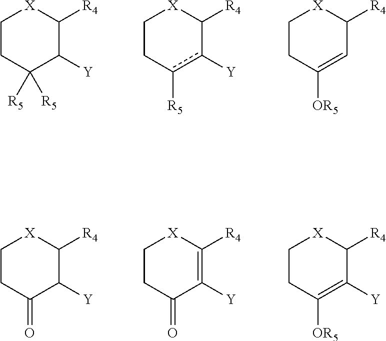

or A is —OR₆ and B is:

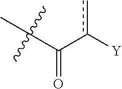

wherein

R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where R_{C} is as previously defined, or CONHR_{E} where R_{E} is as previously defined;

R₅ is H, CO₂R_{C} where R_{C} is as previously defined, or COR_{C}OR_{E} where R_{C} and R_{E} are as previously defined, and where the two R₅ groups are attached to the same group they are the same or different;

R₆ is C₁₋₁₀ alkyl, aryl or arylalkyl;

X is O, N or S;

Y is

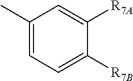

where R_{7A} and R_{7B} are each independently H, OH or OR₇; with the proviso that at least one of R_{7A} and R_{7B} is OH or OR₇;

wherein R₇ is C₁₋₁₀ alkyl, aryl or arylalkyl; and

“ [symbol] ” represents either a single bond or a double bond;

to the central nervous system (CNS), especially to brain tissue, of an individual including the step of administering the compound of general formula (I) to an individual requiring provision of the compound to the CNS, thereby providing the compound to the CNS of the individual, wherein the compound is administered by rectal, vaginal or urethral administration.

In another embodiment, the invention provides a method for providing a compound of general formula (II):

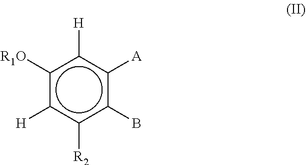

wherein

R₁ is H, or R_{A}CO where R_{A} is C₁₋₁₀ alkyl or an amino acid;

R₂ is H, OH, or R_{B} where R_{B} is an amino acid or COR_{A} where R_{A} is as previously defined;

A and B together with the atoms between them form a six membered ring selected from the group

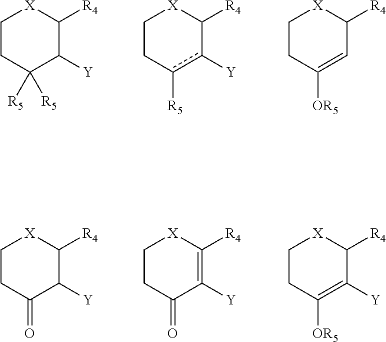

wherein

R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where R_{C} is as previously defined, or CONHR_{E} where R_{E} is as previously defined;

R₅ is H, CO₂R_{C} where R_{C} is as previously defined, or COR_{C}OR_{E} where R_{C} and R_{E} are as previously defined, and where the two R₅ groups are attached to the same group they are the same or different;

X is O, N or S;

Y is

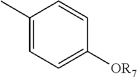

where R₇ is H, or C₁₋₁₀ alkyl; and

“ [symbol] ” represents either a single bond or a double bond;

to the central nervous system (CNS), especially to brain tissue, of an individual including the step of administering the compound of general formula (II) to an individual requiring provision of the compound to the CNS, thereby providing the compound to the CNS of the individual, wherein the compound is administered by rectal, vaginal or urethral administration.

In another embodiment the invention provides a method for providing a compound of general formula (III):

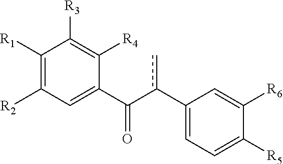

wherein R₁ and R₄ are OR₇; R₂ and R₃ are H or OR₇,

R₅, R₆ are independently H, OH or OR₇,

R₇ is C₁₋₁₀ alkyl, aryl or arylalkyl;

“ [symbol] ” represents either a single bond or a double bond;

to the central nervous system (CNS), especially to brain tissue, of an individual including the step of administering the compound of general formula (III) to an individual requiring provision of the compound to the CNS, thereby providing the compound to the CNS of the individual, wherein the compound is administered by rectal, vaginal or urethral administration.

In another embodiment the invention provides a method for providing a compound of general formula (IV):

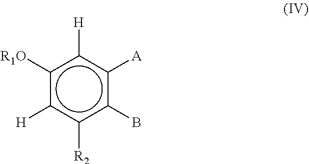

wherein

R₁ is H, or R_{A}CO where R_{A} is C₁₋₁₀ alkyl or an amino acid;

R₂ is H, OH, or R_{B} where R_{B} is an amino acid or COR_{A} where R_{A} is as previously defined;

A and B together with the atoms between them form the group:

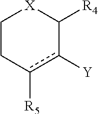

wherein

R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where R_{C} is as previously defined, or CONHR_{E} where R_{E} is as previously defined;

R₅ is substituted or unsubstituted aryl or substituted or unsubstituted heteroaryl;

X is O, N or S;

Y is

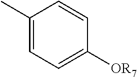

where R₇ is H, or C₁₋₁₀ alkyl; and

“ [symbol] ” represents either a single bond or a double bond;

to the central nervous system (CNS), especially to brain tissue, of an individual including the step of administering the compound of general formula (IV) to an individual requiring provision of the compound to the CNS, thereby providing the compound to the CNS of the individual, wherein the compound is administered by rectal, vaginal or urethral administration.

In another embodiment there is provided a method of treating or preventing a brain cancer in an individual, comprising administering a compound of general formulae (I), (II), (III) or (IV) to an individual requiring said treatment, thereby treating or preventing brain cancer in the individual, wherein the compound is administered by rectal, vaginal or urethral administration.

In another embodiment there is provided a suppository, pessary, intra-urethral device or like formed from a composition described above for use in preventing or treating brain cancer.

In the above described embodiments, the compound of general formulae (I), (II), (III) or (IV) may be rectally administered in an oleaginous base of a type that is typically formulated or used as a suppository for rectal administration of a compound. Typically the oleaginous base comprises a predominance of (>45% w/w base) saturated fatty acids. Preferably the oleaginous base is Theobroma oil (cocoa butter) or an oil fraction or derivative or synthetic version thereof having a saturated fatty acid profile substantially the same as, or identical to the fatty acid profile of Theobroma oil.

The compound of general formulae (I), (II), (III) or (IV) may be at least partially contained or dissolved, preferably completely dissolved in a suppository or pessary base when used in a method described above. In this form, the compound may be bound to hydrophobic compounds of the lipophilic base by hydrophobic interactions.

Further aspects of the present invention and further embodiments of the aspects described in the preceding paragraphs will become apparent from the following description, given by way of example and with reference to the accompanying drawings.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text. All of these different combinations constitute various alternative aspects of the invention.

As described herein, the inventors have rectally administered an isoflavonoid and have observed that the concentration of drug in the plasma at the same time point was around 10-fold less than in the brain at this time point in acutely treated animals, and was undetectable in the plasma at this time point in animals dosed daily for five days. This significant difference between the concentration in the brain tissue and the plasma indicates that the isoflavonoid has entered the brain tissue by crossing the blood-brain barrier. Surprisingly, the outcome appears to be dependent on delivery across rectal mucosa, as it has previously been shown that isoflavonoids are not detectable in brain or spinal cord tissue after oral or IV administration.

Delivery to the CNS and related brain tissue appears to arise from the provision of the isoflavonoid or related compound in the form of a formulation having a substantially hydrophobic or lipophilic base. Further, the formulation is to be provided so as to enable contact with rectal or urogenital mucosa.

Without wanting to be bound by hypothesis, an underlying mechanism of action is believed to involve the hydrophobic association of the isoflavonoid or related compound with fatty acid formulation base and the mucosal uptake of fatty acids administered in the rectal and urogenital spaces. In more detail, when the hydrophobic base of the formulation is liquefied at body temperature in the rectal or urogenital spaces, the isoflavonoid or related compound remains hydrophobically associated (potentially by hydrophobic interactions between the fatty acid chains of the hydrophobic formulation base and the phenolic chemistry of the isoflavonoid or related compound) in the form of a fatty acid/isoflavonoid-type complex. In this condition, a mechanism operating at the rectal or urogenital mucosa for uptake of fatty acid chains may transport the fatty acid/isoflavonoid-type complex across the mucosa whereby the isoflavonoid or related compound is available for therapeutic effect.

It has been generally understood that a critical feature of suppository and pessary formulations and the like is the presence of a base associated with the pharmaceutical active that is selected to enable the partitioning of the base and active. Using the example of a suppository, where the active is hydrophilic, the suppository base is generally hydrophobic or lipophilic, enabling the active to be physically retained in the rectum until the base melts, upon which the active is released for absorption across the mucosa and the base is understood to mix with rectal fluid and to be expelled from the rectal space. In this context the base functions merely as a carrier enabling physical administration of the active.

Partitioning of active and base is very well understood to be essential to the function of a suppository. It is generally accepted that pharmaceutical actives that are highly soluble in a suppository base in fact diffuse much less rapidly out of the base than do those actives which are insoluble or have a low excipient solubility see: Allen L. V in Compounding rectal dosage forms—Part II, Secundum Artem Vol 14 No. 4 Therefore, without partitioning of active and base, when the base (hydrophilic or hydrophobic) melts or otherwise is dissolved in mucosal fluid and expelled from the rectal or urogenital space, the active is dissolved and expelled with the base. It is on the basis of this understanding that hydrophilic actives are generally formulated together with hydrophobic base (typically containing fatty acids, especially saturated fatty acids) and hydrophobic actives are generally formulated with hydrophilic base (for example cyclodextrin etc.)

Notably the invention described herein stands in contrast to these accepted principles of suppository formulation whereby the inventor has recognised that, at least insofar as certain isoflavonoids and related compounds described herein are concerned, there is a surprising advantage that pertains to utilising a hydrophobic or lipophilic base, enabling the dissolution of isoflavonoids and related compounds therein, and from which these isoflavonoids and related compounds would be expected to diffuse less rapidly and therefore to exhibit lower partitioning. As stated, according to this invention it is believed that it is important that the active should not readily diffuse from the fatty acid base as otherwise this would mean a lesser likelihood of transfer of isoflavonoid or related compound across the rectal or urogenital mucosa on uptake of the fatty acid chains.

On the basis of the findings described herein, the inventor has discovered that isoflavonoids and related compounds, when formulated with an oleaginous base and administered rectally, vaginally or through urethral application, cross the blood brain barrier to reach the central nervous system, when previously they could not.

A method that enables these compounds to be provided to the central nervous system may allow for enhanced efficacy in general, and may also allow for treatment of diseases and conditions such as cancer and other conditions.

The inventor has recognised the potential of isoflavonoids and related compounds for treatment of brain cancer or sensitisation of brain cancer cells to chemo- or radiotherapy, and other conditions, when given rectally in the form of a formulation having a hydrophobic or lipophilic base.

In a preferred embodiment, the compound of general formula (II) is selected from the group consisting of

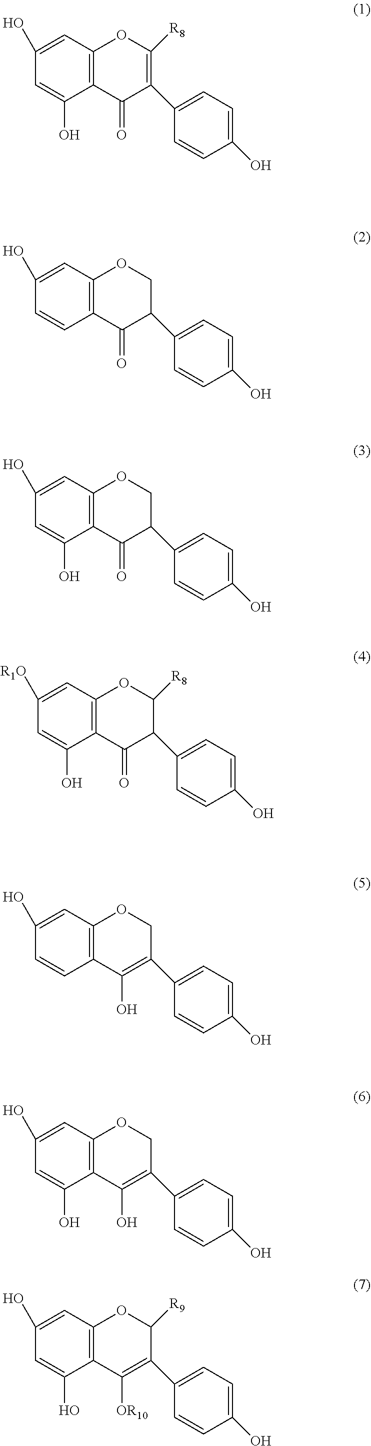

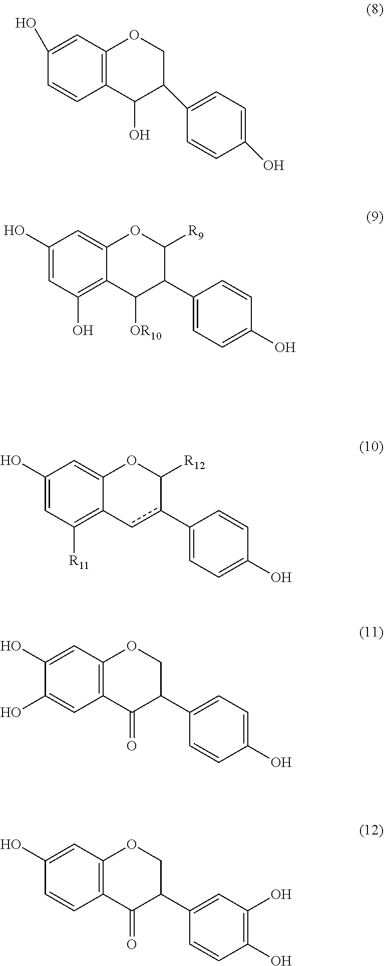

wherein

R₈ is H or COR_{D} where R_{D} is as previously defined;

R₉ is CO₂R_{C} or COR_{E} where R_{C} and R_{E} are as previously defined;

R₁₀ is COR_{C} or COR_{C}OR_{E} where R_{C} and R_{E} are as previously defined;

R₁₁ is H or OH;

R₁₂ is H, COOH, CO₂R_{C} where R_{C} is as previously defined, or CONHR_{E} where R_{E} is as previously defined; and

“ [symbol] ” represents either a single bond or a double bond.

Even more preferably, the compound is selected from:

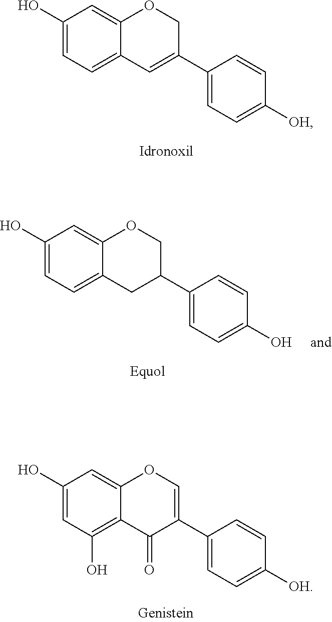 [SMILES: Oc1ccc(C2=Cc3ccc(O)cc3OC2)cc1 ; Oc1ccc(C2COc3cc(O)ccc3C2)cc1 ; "and" ; O=c1c(-c2ccc(O)cc2)coc2cc(O)cc(O)c12]

Even more preferably, the compound is:

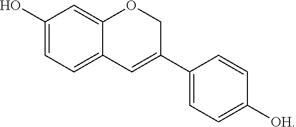 [SMILES: Oc1ccc(C2=Cc3ccc(O)cc3OC2)cc1]

In a preferred embodiment, the compound of general formula (III) is selected from the group consisting of:

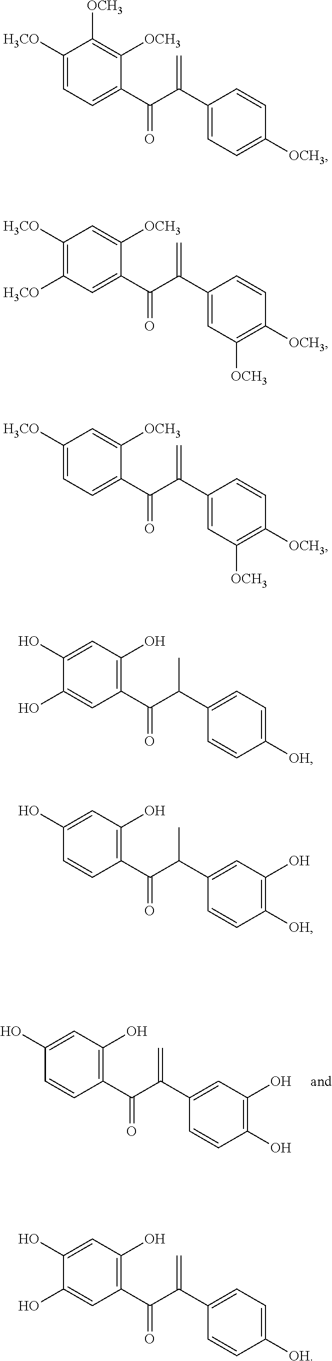

Preferably, the compound is selected from

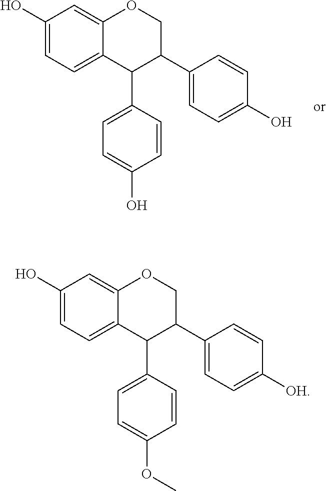 [SMILES: Oc1ccc(C2COc3cc(O)ccc3C2c2ccc(O)cc2)cc1 ; COc1ccc(C2c3ccc(O)cc3OCC2c2ccc(O)cc2)cc1]

As used herein the term “alkyl” refers to a straight or branched chain hydrocarbon radical having from one to ten carbon atoms, or any range between, i.e. it contains 1, 2, 3, 4, 5, 6, 7, 8, 9 or 10 carbon atoms. The alkyl group is optionally substituted with substituents, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “C₁₋₁₀ alkyl” refers to an alkyl group, as defined above, containing at least 1, and at most 10 carbon atoms respectively, or any range in between (e.g. alkyl groups containing 2-5 carbon atoms are also within the range of C₁₋₁₀).

Preferably the alkyl groups contain from 1 to 5 carbons and more preferably are methyl, ethyl or propyl.

As used herein, the term “aryl” refers to an optionally substituted benzene ring. The aryl group is optionally substituted with substituents, multiple degrees of substitution being allowed.

As used herein, the term “heteroaryl” refers to a monocyclic five, six or seven membered aromatic ring containing one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl and substituted versions thereof.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterised and tested for biological activity.

The terms “optionally substituted” or “may be substituted” and the like, as used throughout the specification, denotes that the group may or may not be further substituted, with one or more non-hydrogen substituent groups. Suitable chemically viable substituents for a particular functional group will be apparent to those skilled in the art.

Examples of substituents include but are not limited to:

C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aminosulfonyl, acyloxy, alkoxycarbonyl, nitro, cyano or halogen.

The term “isoflavonoid” as used herein is to be taken broadly and includes isoflavones, isoflavenes, isoflavans, isoflavanones, isoflavanols and similar or related compounds. Some non-limiting examples of isoflavonoid core structures are shown below:

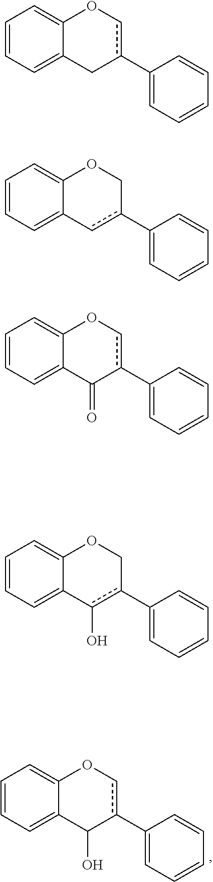

wherein “ [symbol] ” represents either a single bond or a double bond.

Some of the compounds discussed above may be referred to by the names genistein (compound 1 where R₈ is H), and equol and dehydroequol (compound 10 when R₁₁ and R₁₂ is H). Dehydroequol is also known as idronoxil and phenoxodiol.

The phrase “related compounds” when associated with the isoflavonoids are compounds with a similar structure to the isoflavonoids, differing in that the central ring of the isoflavonoid is open. Such compounds are known as aryl di-substituted propanone compounds and have a general backbone according to the structure below:

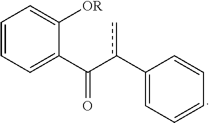

Methods for synthesis of the above described compounds are described in WO1998/008503, WO2005/049008, WO2000066576, and WO2010054438 and references cited therein towards the synthesis, the contents of which are incorporated herein by reference in entirety.

A. Bases for Forming Suppository, Pessary or Urethral Devices

In the disclosure below, ‘base’ may refer to a substance commonly used as a carrier in a suppository, pessary or intra-urethral device.

Generally the base has a solvent power for the isoflavonoid or related compound enabling at least partial, preferably complete dissolution of the isoflavonoid or related compound in the base.

The base may be comprised of, or consist of an oil or fat.

In one embodiment the base includes saturated fatty acids in an amount of 50 to 65% w/w base. Stearic acid may be included in an amount of 25 to 40% w/w base. Palmitic acid in an amount of 25 to 30% w/w base. Longer chain saturated fatty acids such as myristic, arachidic and lauric acid may be included in an amount of <2% w/w base.

Further described herein, it has been found that oleaginous bases that are high in unsaturated fatty acids tend to be less advantageous in the invention. Typically, the oleaginous base includes unsaturated fatty acids in an amount of 35 to 50% w/w base. Monounsaturated fatty acid may be included in an amount of 30 to 45% w/w base. Oleic acid may be included in an amount of 30 to 40% w/w base. Polyunsaturated fatty acids such as linoleic and alpha linolenic acid may be included in an amount of 0 to 5% w/w base.

Theobroma oil (cocoa butter) has been a traditional base in a suppository because of: (a) its non-toxic and non-irritant nature, and (b) its low melting point, meaning that it readily dissolves at body temperature when placed within a bodily cavity, However, it is increasingly being replaced for a number of reasons. One reason is its variability in composition, a consequence of its natural origins; theobroma oil also is polymorphic, meaning it has the ability to exist in more than one crystal form. Another is that the formulated product needs to be kept refrigerated because of its low melting point, rendering it unsuitable in tropical regions. This has led to a number of substitute products offering a range of advantages over theobroma oil such as greater consistency, decreased potential for rancidity, and greater ability to tailor phase transitions (melting and solidification) to specific formulation, processing, and storage requirements.

Nevertheless, theobroma oil or a fatty base with similar composition and physico-chemical properties has been found to be a preferred embodiment of the invention.

Typically the oleaginous base comprises a predominance of (>45% w/w base) of saturated fatty acids. Preferably the oleaginous base is Theobroma oil (cocoa butter) or an oil fraction or derivative or synthetic version thereof having a saturated fatty acid profile substantially the same as, or identical to the fatty acid profile of Theobroma oil.

Other examples of oils that may be used to provide or obtain fatty acids useful as bases include those obtainable from natural sources such as canola oil, palm oil, soya bean oil, vegetable oil, and castor oil. Oils derived from these sources may be fractionated to obtain oil fractions containing saturated fatty acids.

The base may be formed or derived from a hard fat, butter or tallow.

A base may comprise esterified or non-esterified fatty acid chains. The fatty acid chains may be in the form of mono, di and trigycerides, preferably of saturated fatty acid chains of C9-20 chain length.

A suppository base may be formed from synthetic oils or fats, examples including Fattibase, Wecobee, Witepesoll (Dynamit Nobel, Germany), Suppocire (Gatefosse, France, Hydrokote and Dehydag.

The proportion of the oleaginous suppository base in the final product is a function of the dosage of active pharmaceutical ingredient and the presence of other pharmaceutical or inert ingredient (if any) but may be provided by way of example in an amount of about 1 to 99% w/w formulation.

In one embodiment the lipophilic suppository base contains fatty acids and wherein 50 to 100% of the fatty acids of the base are saturated fatty acids, preferably, 90 to 99% of the fatty acids of the base are saturated fatty acids. 30 to 60%, preferably about 40% of fatty acids of the base may be stearic acid. 20 to 30%, preferably about 25% of fatty acids of the base may be palmitic acid. 15 to 25%, preferably about 20% of fatty acids of the base may be lauric acid. 5 to 10%, preferably about 8% of fatty acids of the base may be myristic acid.

B. Manufacture

The suppository, pessary and devices for urethral application of the invention containing an isoflavonoid or related compound may be prepared as follows. The isoflavonoid or related compound is contacted with a suppository base (as described above) in molten form in conditions enabling at least partial, preferably complete or substantially complete dissolution of the isoflavonoid or related compound in the base. This solution is then poured into a suitable mould, such as a PVC, polyethylene, or aluminium mould. For example, the isoflavonoid or related compound may be contacted with the base at a temperature of from about 35° C. to about 50° C. and preferably from about 40° C. to about 44° C. The isoflavonoid or related compound can be milled or sieved prior to contact with the base.

In one embodiment, the conditions provided for manufacture, and formulation or device formed from same, enable at least, or provide at least, 50%, preferably 60%, preferably 70%, preferably 80%, preferably 90%, preferably 95% of the isoflavonoid for a given dosage unit to be dissolved in the dosage unit. In these embodiments, no more than 50% of the isoflavonoid or related compound for a given dosage unit, preferably no more than 40%, preferably no more than 30%, preferably no more than 20%, preferably no more than 10%, preferably no more than 5% of isoflavonoid or related compound for a given dosage unit may be in admixture with, (i.e. undissolved in) the suppository base of the dosage unit.

In a preferred embodiment, all of the isoflavonoid or related compound added to a dosage unit is dissolved in the base. In this embodiment, no isoflavonoid or related compound is left in admixture with the suppository base. This is believed to increase the likelihood of the uptake of all of the isoflavonoid or related compound given in the dosage unit.

It will be understood that the objective of the manufacture process is not to admix, or to mingle, or to blend the suppository base with the isoflavonoid or related compound as generally occurs in pharmacy practice of admixing components, as it is believed that the resulting admixture would have a lower likelihood of providing therapeutic benefit. In this context, it is particularly important that any other excipient, carrier or other pharmaceutical active does not interfere with the dissolution of the isoflavonoid or related compound in the base, for example as may occur if the isoflavonoid or related compound forms a complex with a charged molecular species (other pharmaceutical active, carrier or excipient), the result of which would be to decrease the propensity of the complex, and therefore the isoflavonoid or related compound contained in it, to dissolve in the suppository base.

Optionally the suppositories, pessaries or intra-urethral devices may be coated, prior to packing, for example with cetyl alcohol, macrogol or polyvinyl alcohol and polysorbates to increase disintegration time or lubrication or to reduce adhesion on storage.

One or more sample suppositories, pessaries, or intra-urethral devices from each batch produced are preferably tested by the dissolution method of the present invention for quality control. According to a preferred embodiment, a sample from each batch is tested to determine whether at least about 75 or 80% by weight of the base dissolves within 2 hours.

Typically the suppository, pessary or like device according to the invention is substantially hydrophobic or lipophilic throughout and does not contain a hydrophilic substance such as hydrophilic carrier or pharmaceutical active, or hydrophilic foci or region formed from the ligation or complexing of the isoflavonoid or related compound to or with another pharmaceutical compound, carrier or excipient.

Preferably the formulation for forming the suppository, pessary and devices for urethral application does not include a further pharmaceutical active, cytotoxic or chemotherapeutic agent. In this embodiment, the only active is the isoflavonoid or related compound and the formulation does not include a platin, taxane or other cytotoxic or chemotherapeutic agent.

C. Physical Characteristics

The total weight of the suppository preferably ranges from about 2250 to about 2700 mg and more preferably from about 2250 to about 2500 mg. According to one embodiment, the suppository has a total weight ranging from about 2300 mg to about 2500 mg.

The suppository or pessary is preferably smooth torpedo-shaped.

The melting point of the suppository or pessary is generally sufficient to melt in the patient's body, and is typically no more than about 37° C.

In one particularly preferred embodiment there is provided:
a kit including:

a plurality of suppositories sufficient in number to provide an individual with a suppository once daily, or twice daily, for a period of 30 to 90 days, preferably 30 to 60 days, preferably 30 days

each suppository including:
400 mg or 800 mg of idronoxil;
a suppository base in the form of cocoa butter;

wherein the suppository base in provided an amount of 1-99% w/w of the suppository,
the kit further including:

written instructions to provide the suppository once daily, or twice daily for a period of 30 to 90 days, preferably 30 to 60 days, preferably 30 days, preferably for use in treatment of cancer, more preferably for sensitising cancer cells to cytotoxic effect of a chemo- or radiotherapy, preferably where the cancer is prostate cancer.

D. Providing Compounds to CNS

As described herein, the blood-brain barrier prevents the passive diffusion of many exogenous or xenobiotic compounds from the blood to the CNS. This barrier enables the passive diffusion of a limited number of compounds (water, gases and certain small molecular weight compounds) while most other compounds are subject to active transport, requiring dedicated transporters. Many compounds given systemically have been found not to cross the blood-brain barrier, and in each example, the inference is that the relevant compound does not have a suitable transport system enabling translocation of the compound across the blood-brain barrier. This failure to cross the blood-brain barrier has limited the value of those compounds in treatment of CNS injury, diseases or related conditions that have otherwise been found to have useful pleotropic effects in vivo. This is a critical and significant issue for healthcare, because it restricts the amount of reagents that can be given systemically for treatment of brain and related CNS disorders. For example, temozolomide is perhaps the only anti-neoplastic agent that is useful for treatment of multiforme glioblastoma, and this is because temozolomide is the only anti cancer drug shown to cross the blood-brain barrier. In the circumstances, the finding by the inventors that certain phenolic compounds can be made to permeate the brain tissue by implementation of the methods and compositions herein is highly significant because it enables a wide range of beneficial activities, such as cytotoxic, apoptotic or cell survival signals to be provided to the CNS by molecules that had previously been considered unsuited to the purpose.

Whilst the present invention relates to isoflavonoids and related compounds, it is envisaged that the administration of other insoluble drugs, (including phenolic compounds and benzopyrans), formulated in a similar manner for rectal, vaginal or urethral application will also provide an improved access to the central nervous system. In particular it is envisaged that by formulating these drugs in a lipophilic base so that the drugs substantially or completely dissolve in the base, and therefore do not partition from the base when the suppository melts at body temperature, it will be possible to have these drugs transfer across the blood-brain barrier as observed for certain isoflavones in the work described herein. One example of such compounds or drugs that are not isoflavonoids to which the work described herein may apply are non-steroidal anti-inflammatory drugs (NSAIDs) such as ibuprofen, flurbiprofen, aspirin and indomethacin, and other phenolic drugs such as paracetamol.

Given above it is anticipated that further to delivery of isoflavonoids to the brain and CNS it is believed that the invention is applicable to the delivery of other compounds to the CNS and brain, especially compounds that have low aqueous solubility, that have been shown to be unable to cross the blood-brain barrier when given orally or by IV and that are generally soluble in a lipophilic base so as not to partition from the base when the base is liquefied. Thus in another embodiment there is provided a method of providing a therapeutic compound to the brain or CNS including providing a composition including a lipophilic base and a therapeutic compound in which the therapeutic compound is partially or completely dissolved in the base, and administering the composition via the rectum, vagina or urethral routes as described herein in relation to the administration of isoflavonoids such as idronoxil. Preferably the therapeutic compound has low aqueous solubility. More preferably the compound comprises one or more phenol-containing groups. Still more preferably the compound is a benzopyran.

The studies herein show a 10 fold increase in isoflavonoid content in brain tissue compared with plasma. This clearly demonstrates that isoflavonoids, when formulated and delivered according to the method of the invention are capable of crossing the blood-brain barrier. While not wanting to be bound by hypothesis, these results indicate that at least some of the CNS uptake of the isoflavonoid arises from passive diffusion across the blood-brain barrier, although it is possible that active transport of a compound may result in at least some of the isoflavonoid crossing the blood-brain barrier.

Importantly, the studies herein show that the isoflavonoid formulated and administered according to the invention is retained in the CNS and brain tissue after daily dosing of the isoflavonoid for a period of 5 days. This is important because it demonstrates that it is possible to expose CNS tissue to isoflavonoid across a desired dosing schedule, enabling the drug to be presented for the period of time required for treatment or resolution of the relevant disease, disorder or condition. The finding is significant because some drugs that have been shown to cross the blood-brain barrier are either metabolised or actively effluxed from the CNS and therefore have a relatively short half-life that precludes the ability of those drugs to treat a CNS disease or disorder.

A further important finding is that the isoflavonoid formulated and administered according to the invention does not accumulate or build up in the CNS or brain tissue suggesting either a passive or active mechanism enabling the isoflavonoid to reach a steady state. This result is seen in the study where isoflavonoid is given daily for a period of 5 days with no increase in amount or accumulation of isoflavonoid from day 1 to day 6.

A number of assays are available for demonstrating the transfer of an isoflavonoid or other aqueous insoluble phenolic compound across the blood-brain barrier to brain and CNS tissue. A liquid chromatography-mass spectrometry (LC-MS) experimental procedure is described in the examples that follow.

In an alternative approach, a compound of interest may be ¹⁴C labelled and penetration of the blood-brain barrier determined by quantitative whole body autoradiography. This approach enables a study of drug location at various time points including 1, 6, 24, 72, 120 and 168 hours.

A number of options are described for ex vivo assessment of brain penetration, including assessment of in situ brain perfusion which provides a kinetic measure of the uptake of a compound into the brain and calculation of brain/plasma ratio, which provides a simple ratio of drug concentration in brain and plasma useful for calculating an apparent permeability coefficient. See Alavijeh et al 2005.

Approaches for in vivo assessment of brain penetration are also described (Alavijeh et al 2005) which consider a full pharmacokinetic profile including Cmax, half-life and AUC. For example, ventricular CSF may provide a good measure of free drug concentration that correlates with brain interstitial concentrations. Ventricular CSF may be sample post mortem or in vivo. Tissue microdialysis permits measurement of the concentration of a compound in brain interstitial fluid over time, for calculation of Cmax, half-life and AUC.

E. Methods of Treatment

The formulations according to the invention in suppository, pessary, intra-urethral device or like form are useful for improving the bioavailability of isoflavonoids and related compounds in the central nervous system, including brain tissue and parenchyma, spinal cord, ventricles, sub arachnoid space and related meningeal tissue and the cerebrospinal fluid.

In one particularly preferred embodiment, the compositions are useful for treatment of brain cancer, whereby the isoflavonoid or related compound is used as a cytotoxic monotherapy, or as a chemo-sensitising agent for another cytotoxic molecule.

Thus in one embodiment there is provided a method of treating or preventing brain cancer in an individual, including administering to a person in need thereof a suppository, pessary or intra-urethral device formed from a composition including a compound of Formula (I), (II), (III) or (IV).

In one embodiment there is provided a use of a compound of Formula (I), (II), (III) or (IV) in the preparation of a suppository, pessary or intra-urethral device for the prevention or treatment of brain cancer.

In another embodiment there is provided a suppository, pessary or intra-urethral device formed from a composition including a compound of Formula (I), (II), (III) or (IV) for use in preventing or treating brain cancer.

Methods for applying a suppository are well known in the art. Generally the methods involve inserting the suppository to a point aligned with the inferior and medial haemorrhoid veins, thereby enabling the release of the drug to the inferior vena cave.

Methods for applying a pessary, or for urethral application of a pharmaceutically active ingredient are well known in the art.

‘Treatment’ generally refers to both therapeutic treatment and prophylactic or preventative measures.

Subjects requiring treatment for cancer include those already having a benign, pre-cancerous, or non-metastatic tumor as well as those in which the occurrence or recurrence of cancer is to be prevented.

The objective or outcome of treatment may be to reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying disease progression.

In one embodiment, the method is particularly useful for extending survival of the human, including overall survival as well as progression free survival.

In one embodiment, the method is particularly useful for providing a complete response to therapy whereby all signs of cancer in response to treatment have disappeared. This does not always mean the cancer has been cured.

In one embodiment, the method is particularly useful for providing a partial response to therapy whereby there has been a decrease in the size of one or more tumors or lesions, or in the extent of cancer in the body, in response to treatment.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is pre-cancerous or pre-neoplastic.

In one embodiment, the cancer is a secondary cancer or metastases.

“Brain cancer” or “brain tumor” include all cancers and tumors inside the cranium or in the central spinal canal. They may be created by an abnormal and uncontrolled cell division, normally either in the brain itself (neurons, glial cells (astrocytes, oligodendrocytes, ependymal cells, myelin-producing Schwann cells, lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to, oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma (e.g., pilocystic astrocytoma, anaplastic astrocytoma), polar spongioblastoma, astroblastoma, gliomatosis cerebri, pituitary tumor, primary lymphoma of the brain, primary germ cell tumor of the brain, choroid plexus papilloma, acoustic neuroma, craniopharyngioma, nerve glioma, a primitive neuroectodermal tumor, and rhabdoid tumor.

In certain embodiments, the cancers and neoplastic diseases of the brain or CNS may potentially respond to EGFR, HER2, VEGFR2, or Src family kinase inhibitors.

In a particularly preferred emboidmment, the brain or central nervous system tumor may be a glioblastoma, e.g., glioblastoma multiforme.

In some embodiments, the invention provides a method for treating refractory tumors, particularly refractory malignant tumors. Refractory tumors include tumors that fail or are resistant to treatment with chemotherapeutic agents alone, radiation alone or combinations thereof. For the purposes of this specification, refractory tumors also encompass tumors that appear to be inhibited by treatment with chemotherapeutic agents and/or radiation but recur up to five years, sometimes up to ten years or longer after treatment is discontinued.

In some embodiments, the central nervous system or brain tumor is a tumor that is responsive to treatment with trastuzumab. In some embodiments, the central nervous system or brain tumor is a tumor that is responsive to treatment with an intracellular or extracellular HER2 antagonist. In some embodiments, the central nervous system or brain tumor is a tumor that is responsive to treatment with an intracellular or extracellular EGFR antagonist. Responsiveness to an extracellular or intracellular antagonist of EGFR or HER2 includes responsiveness in an in vitro assay, for example for an antagonist that does not cross the BBB.

In the above described embodiments, the formulation according to the invention may be useful for preventing doubling time of the cancer cells or otherwise inhibiting tumour growth, either through cytotoxic effect on the tumour cells or otherwise by generally inhibiting cell replication. In these embodiments it will be understood that the suppository formulation provides an anti neoplastic “monotherapy” effect.

In another embodiment, the method of treatment described above further includes the step of administering cytotoxic chemotherapy or radiotherapy to the individual.

In yet another embodiment there is provided a method of sensitising a brain cancer to chemo or radiotherapy including the steps of:
providing an individual having a brain cancer in need of chemo or radiotherapy;
administering to the individual a suppository, pessary or intra-urethral device formed from a formulation according to the invention;
administering chemo or radio-therapy to the individual.

In another embodiment, the treatment provides for sensitisation of the brain tumour to radiotherapy, especially stereotactic radiotherapy. In one embodiment the treatment may provide for a reduction in tumour size utilising a sub-optimal radiation dose. It will be understood that a suboptimal radiation dose is one incapable of reducing tumour size in the absence of isoflavonoid formulation treatment.

In another embodiment, the treatment provides for sensitisation of the brain tumour to chemotherapy. In one embodiment, the treatment provides for a reduction in tumour size utilising a sub-optimal chemotherapy dose. It will be understood that a suboptimal chemotherapy dose is one incapable of reducing tumour size in the absence of isoflavonoid formulation treatment.

In one embodiment, the treatment containing the isoflavonoid or related compound is provided either as a cytotoxic monotherapy, or as a radio or chemosensitising therapy according to a variable dosing regime, prior to, or at the time of radio or chemotherapy. The variable dosing regimen may include an increasing dose of isoflavonoid treatment during a run in period prior to radio or chemotherapy and/or an increasing dose during radio or chemotherapy. In one example, the isoflavonoid or related compound is provided in a dose of about 400 mg once daily for a period of 1 to 2 weeks and increased to 800 mg once daily for a period of 1 to 2 weeks or 1 month or longer, and further increased to 1600 mg (2×800 mg) once daily for a period of 1 to 2 weeks or 1 month or longer. Actual amounts will be influenced by disease status, age, weight, gender and other pharmacologically relevant variables.

In some embodiments the compound of Formulae 1 to IV, and particularly idronoxil, or one of the following compounds:

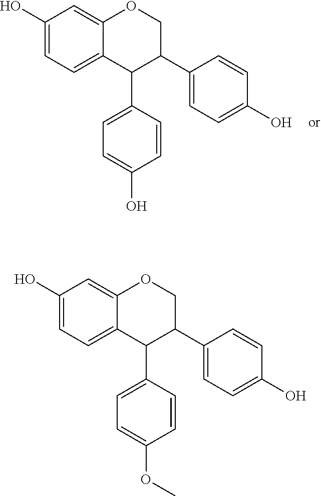 [SMILES: Oc1ccc(C2COc3cc(O)ccc3C2c2ccc(O)cc2)cc1 ; COc1ccc(C2c3ccc(O)cc3OCC2c2ccc(O)cc2)cc1]

is administered according to the methods and compositions of this invention described above with temozolomide. Thus there is provided a method for treatment of an individual for brain cancer, in particular for treatment of glioblastoma multiforme, wherein the individual has received, or is to receive temozolomide, the method including the step of rectally administering a suppository formulation described herein including idronoxil or one of the following compounds

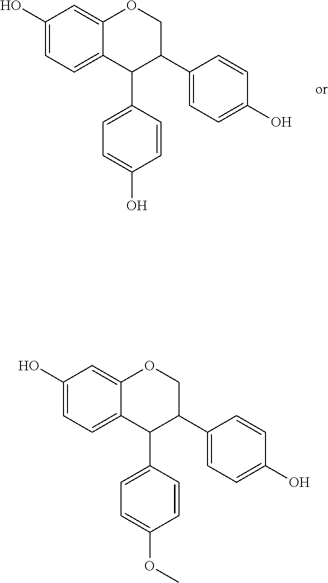 [SMILES: Oc1ccc(C2COc3cc(O)ccc3C2c2ccc(O)cc2)cc1 ; COc1ccc(C2c3ccc(O)cc3OCC2c2ccc(O)cc2)cc1]

to the individual.

In these embodiments, these compounds may be administered for the treatment of adult patients with newly diagnosed glioblastoma multiforme concomitantly with radiotherapy and then maintenance treatment with temozolomide. In such embodiments, temozolomide may be administered at 75 mg/m2 for 42 days concomitant with focal radiotherapy (e.g., 60 Gy administered in 30 fractions) followed by an initial maintenance dose of 150 mg/m2 once daily for Days 1-5 of a 28-day cycle of temozolomide for 6 cycles.

In some embodiments where the compound of Formulae 1 to IV, and particularly idronoxil, is administered with temozolomide, the compound of Formula A, the administration may be for the treatment of adult patients with refractory anaplastic astrocytoma, i.e., patients who have experienced disease progression on a drug regimen containing nitrosourea and procarbazine. In such embodiments, temozolomide may be administered at an initial dose of 150 mg/m2 once daily for 5 consecutive days per 28-day treatment cycle.

Temozolomide may be administered in unit dosage form as, e.g., 5 mg, 20 mg, 100 mg, 140 mg, 180 mg, or 250 mg capsules or as a 100 mg powder for injection (e.g., as an intravenous infusion over 90 minutes)

For patients with newly diagnosed high grade glioma, temozolomide may be administered in the concomitant phase at 75 mg/m2 daily for 42 days concomitant with focal radiotherapy (60 Gy administered in 30 fractions) followed by maintenance temozolomide for 6 cycles. For the maintenance phase, temozolomide may be administered as follows: Cycle 1: Four weeks after completing the temozolomide plus radiotherapy phase, temozolomide is administered for an additional 6 cycles of maintenance treatment. Dosage in Cycle 1 (maintenance) is 150 mg/m2 once daily for 5 days followed by 23 days without treatment. Cycles 2-6: At the start of Cycle 2, the dose can be escalated to 200 mg/m2, if the CTC nonhematologic toxicity for Cycle 1 is Grade less than or equal to 2 (except for alopecia, nausea, and vomiting), absolute neutrophil count (ANC) is greater than or equal to 1.5×109/L, and the platelet count is greater than or equal to 100×109/L. The dose remains at 200 mg/m2 per day for the first 5 days of each subsequent cycle except if toxicity occurs. If the dose was not escalated at Cycle 2, escalation should not be done in subsequent cycles.

For adult patients with refractory anaplastic astrocytoma, the initial dose of temozolomide is 150 mg/m2 once daily for 5 consecutive days per 28-day treatment cycle.

For additional guidance as to dosing of temozolomide, the prescribing information for TEMODAR® (the brand name temozolomide sold by Merck & Co., Inc.) may be consulted.

The efficacy of an isoflavonoid, in particular indronoxil, genestien and others for killing tumor cells is well established in the literature (See Brown et al. 2008).

A number of pre-clinical animal models for assessing the efficacy of a lead compound for killing tumors of the CNS and brain are also known. In one example, human luciferase expressing tumor cells are implanted intracranially. Assessment of tumor growth and burden in the brain is on the basis of detection of bioluminescence.

Utilising the aforementioned model, a compound of Formulae I to IV, particularly idronoxil may be given rectally according to the invention to mice having the implant, about 8 days after the implant. The bioluminescence may then be detected at time points during which isoflavonoids have been found in CNS and brain tissue as per the studies according to this invention.

The model may also include tail vein treatments of temozolomide or other chemotherapeutics to assess the increase in potency of these drugs when an isoflavonoid is delivered in the model according to the methods and compositions of this invention.

The model may further include irradiation, either total body or cranial specific to assess an increased sensitivity of tumor cells to radiation arising from delivery of isoflavanoid according to the methods and compositions of this invention.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1 Acute and 5-Day Repeated Rectal Dose Study in Sprague Dawley Rats Followed with a Distribution Investigation in Brain Tissue and Blood

The purpose of this study was to determine the absorption of Idronoxil by rectal administration following acute and repeated administration in various brain tissue and blood in SPF Sprague Dawley male rats.

7.0. Method

TABLE 1
LCMS settings

| Item | | | | | |
|---|---|---|---|---|---|
| Buffers | Buffer A | 2 mM ammonium acetate 20% | | | |
| | | methanol | | | |
| | Buffer B | methanol | | | |
| Pump | Flow rate | 0.4 mLs/min | | | |
| | | Time | | | |
| | Gradient | (min) | % A | % B | Curve |
| | | 0 | 99.0 | 1.0 | Initial |
| | | 0.00 | 99.0 | 1.0 | 6 |
| | | 1.00 | 65.0 | 35.0 | 6 |
| | | 3.40 | 65.0 | 35.0 | 6 |
| | | 3.50 | 25.0 | 75.0 | 6 |
| | | 4.00 | 25.0 | 75.0 | 6 |
| | | 4.10 | 20.0 | 80.0 | 6 |
| | | 4.45 | 0.1 | 99.9 | 6 |
| | | 4.50 | 99.0 | 1.0 | 6 |
| | | 7.00 | 99.0 | 1.0 | 6 |

| | | |
|---|---|---|
| Auto- | Sample temperature | 20° C. |
| sampler | Injection volume | 2 μL |
| | Injection mode | full loop |
| Column | Waters Acquity [symbol] EH | 2.1 × 100 mm, 1.7 μm |
| | C18 | |
| | Temperature | 45° C. |
| M [symbol] | Mode | negative |
| | Detection method | Multiple Reaction Monitoring |
| | Capillary voltage | 1.5 (kV) |
| | Desolvation temperature | 500° C. |
| | Source temperature | 140° C. |
| | Desolvation gas | 1000 (L/Hr) |
| | Conc Gas Flow | 100 (L/Hr) |
| | Collision Gas Flow | 0.17 (mL/min) |
| Idronoxil | Mass (Precursor > | 239.05 > 144.96 |
| | Product ion) | |
| | Conc voltage | 28 |
| | Collision energy | 18 |
| | Mass (Precursor > | 239.05 > 92.92 |
| | Product ion) | |
| | Conc voltage | 28 |
| | Collision energy | 26 |
| Internal | Mass (Precursor > | 347.2 > 277.0 |
| Standard | Product ion) | |
| (Triphendiol) | Conc voltage | 38 |
| | Collision energy | 15 |

[symbol]

indicates data missing or illegible when filed

7.1. LCMS Settings

7.2. Test Item

TABLE 2
Test item

| Name | Batch |
|---|---|
| Idronoxil Suppository | IXL16001 |
| Idronoxil (TI/608) | — |
| Triphendiol (TI/609) | — |
| Daidzein | Sigma P/N D7802-25 g |
| | L/N 085M4116V |

7.3. Preparation of Standards

Calibration standards were prepared in acetonitrile. A total of 8 non-zero standards are used at the following concentrations 0.5-1,000 ng/mL. Stock were prepared and stored for up to 1 day at 2-8° C. Intermediate and working stocks are prepared fresh from stock standard solutions. Internal standard solutions were prepared and stored for up to 1 day at 2-8° C.

7.3.1 Stock Standard Solution

Stock standard solutions were prepared as follows. Prepare a stock solution for preparation of working and calibration and QC standards

TABLE 3
Stock Standard Solutions

| | Standard | | Solvent | Concen- | |
|---|---|---|---|---|---|
| | Weight | Solvent | Volume | tration | Storage/ |
| Name | (mg) | Type | (mL) | (mg/mL) | Expiry |
| Idronoxil | 1 | Methanol | 1 | 1 | 1 day at |
| Stock | | | | | 2-8° C. |
| Triphendiol | 0.5 | Acetonitrile | 1 | 0.5 | 1 day at |
| Stock | | | | | 2-8° C. |
| (Internal | | | | | |
| Standard) | | | | | |
| Daidzein | 0.25 | Methanol | 1 | 0.25 | 1 day at |
| Stock | | | | | 2-8° C. |

7.3.2 Internal Standard

Internal Standard
Prepare “Internal Standard” from the Stock Standard Solution (Table 3) using the following dilution scheme.

TABLE 4
Non-matrix working internal standards

| | | | | | Stock |
|---|---|---|---|---|---|
| | Triphendiol | Daidzein | Solvent | Final | concen- |
| | Stock | Stock | Acetonitrile | Volume | tration |
| Tube | (μL) | (μL) | (μL) | (mL) | (ng/mL) |
| Internal | 40 | 80 | Make up to | 100 | 200 |
| Standard | | | 100 mLs | | |

7.3.3 Working Standard Solution

Non-Matrix
“Non-matrix working solutions” were prepared (Table 5) using the following dilution scheme.

TABLE 5
Non-matrix working standards

| | IDRONOXIL | Stock | Aceto- | Stock |
|---|---|---|---|---|
| Working | Stock | volume | nitrile | Concentration |
| solution | (μL) | (uL) | (μL) | (ng/mL) |
| SC1 | 25 | — | 975 | 25000.0 |
| SC2 | — | 200 of SC1 | 200 | 12500.0 |
| SC3 | — | 100 of SC1 | 900 | 2500.0 |
| SC4 | — | 100 of SC2 | 900 | 1250.0 |
| SC5 | — | 100 of SC3 | 900 | 250.0 |
| SC6 | — | 100 of SC4 | 900 | 125.0 |
| SC7 | — | 100 of SC5 | 900 | 25.0 |
| SC8 | — | 100 of SC6 | 900 | 12.5 |
| Internal | — | — | 200 | — |
| Standard | | | | |
| Solvent | — | — | 200 | — |
| blank | | | | |

7.3.4 Non-Matrix Working Quality Control Standard Solution

Prepare “Non-matrix working solutions” from the Stock solution (Table 3) using the following dilution scheme.

TABLE 6
Non-matrix working QC standards

| | IDRONOXIL | Stock | Aceto- | Stock |
|---|---|---|---|---|
| Working | Stock | volume | nitrile | Concentration |
| solution | (μL) | (uL) | (μL) | (μ/mL) |
| SQCH | 15 | | 985 | 15000.0 |
| SQCM | — | 200 of SQCH | 800 | 3000.0 |
| SQCL | — | 100 of SQCM | 900 | 300.0 |

7.3.5. Preparation of Calibration Standards

Calibration standards were prepared by mixing the non-matrix working standard (Table 5) with internal standard and water as follows.

TABLE 7
Calibration standards

| | Stock | | | Internal | Concen- |
|---|---|---|---|---|---|
| Working | volume | Acetonitrile | Water | Standard | tration |
| solution | (uL) | (μL) | (μL) | (μL) | (ng/mL) |
| C1 | 20 of SC1 | — | 80 | 400 | 1000.0 |
| C2 | 20 of SC2 | — | 80 | 400 | 500.0 |
| C3 | 20 of SC3 | — | 80 | 400 | 100.0 |
| C4 | 20 of SC4 | — | 80 | 400 | 50.0 |
| C5 | 20 of SC5 | — | 80 | 400 | 10.0 |
| C6 | 20 of SC6 | — | 80 | 400 | 5.0 |
| C7 | 20 of SC7 | — | 80 | 400 | 1.0 |
| C8 | 20 of SC8 | — | 80 | 400 | 0.5 |
| Internal | — | 20 | 80 | 400 | — |
| Standard | | | | | |
| Solvent | — | 20 | 80 | 400 | — |
| blank | | | | | |

7.3.6. Preparation of Quality Control Samples

Quality control standards were prepared by mixing the non-matrix working QC standard (Table 6) with internal standard and water as follows.

TABLE 8
Quality Control standards

| | Stock | | | Internal | Concen- |
|---|---|---|---|---|---|
| Working | volume | Acetonitrile | Water | Standard | tration |
| solution | (uL) | (μL) | (μL) | (μL) | (ng/mL) |
| QCH | 20 of SQCH | — | 80 | 400 | 600.0 |
| QCM | 20 of SQCM | — | 80 | 400 | 120.0 |
| QCL | 20 of SQCL | — | 80 | 400 | 12.0 |

7.4. In-Life Experimental

7.4.1. Dosing and Sampling Schedule

Rectal: 50 mg/mL from the tip of a 2 g suppository was administered to each rat. A new suppository was used each day.

Group 1 animals were used as control. The animals in Group 2 were dosed once, bled at 30 and 120 minutes following test item administration using the tail vein. Twenty four (24) hours following test item administration, blood was collected the animals were sacrificed and organs were dissected, weighed and extracted to determine the test item concentration. The animals in Group 3 were dosed daily for 5 times and sacrificed on Day 6. They were bled using the tail vein 30 and 120 minutes following test item administration and twenty four (24) hours following test item administration, blood was collected the animals were sacrificed and organs were dissected, weighed and extracted to determine the test item concentration. Urine was collected at 2 hours for 15-30 minutes.

TABLE 9
Group 1 - Control

| Animal | | |
|---|---|---|
| Number | Administration | Bleeding Method |
| R5696 | No treatment | Cardiac puncture |
| R5697 | No treatment | Cardiac puncture |

TABLE 10
Group 2 - Acute Study (one Administration only)

| | | | Sampling | | | |
|---|---|---|---|---|---|---|
| | | | time after | | | |
| | | | dosing | | Sampling | Bleeding |
| | | | (mins)- | | time after | Method - |
| Animal | Route of | Dose | Conscious | Bleeding | dosing | Animals were |
| Number | administration | (mg) | animals | Method | (hrs) | sacrificed |
| R5698 | Rectal | 50 | 30, 120 | Tail | 24 | Cardiac |
| | | | | | | puncture |
| R5699 | Rectal | 50 | 30, 120 | Tail | 24 | Cardiac |
| | | | | | | puncture |
| R5700 | Rectal | 50 | 120 | Tail | 24 | Cardiac |
| | | | | | | puncture |
| R5701 | Rectal | 50 | 120 | Tail | 24 | Cardiac |
| | | | | | | puncture |

TABLE 11
Group 3 - 5-Day Repeated Dose Study
Blood and urine samples were collected on Day 1 and Days 5 and 6 (Sacrifice).
Blood and urine samples were collected on Day 1 and Days 6 and 6 (Sacrifice).

| | | | Sampling | | | | |
|---|---|---|---|---|---|---|---|
| | | | time after | | | | |
| | | | dosing | | | Sampling | |
| | | | (hrs) - | | Urine | time after | |
| Animal | Route of | Dose | conscious | Bleeding | Collection | dosing | Bleeding |
| Number | Administration | (mg) | animals | Method | (hrs) | (hrs) | Method |
| R5702 | Rectal | 50 | 0.5, 2 | Tail | 2 | 24 | Day 1 tail/Day 6 |
| | | | | | | | Cardiac puncture |
| R5703 | Rectal | 50 | 0.5, 2 | Tail | 2 | 24 | Day 1 tail/Day 6 |
| | | | | | | | Cardiac puncture |
| R5704 | Rectal | 50 | 0.5, 2 | Tail | 2 | 24 | Day 1 tail/Day 6 |
| | | | | | | | Cardiac puncture |
| R5705 | Rectal | 50 | 0.5, 2 | Tail | 2 | 24 | Day 1 tail/Day 6 |
| | | | | | | | Cardiac puncture |
| R5706 | Rectal | 50 | 0.5, 2 | Tail | 2 | 24 | Day 1 tail/Day 6 |
| | | | | | | | Cardiac puncture |

7.4.2 Bleeding/Necropsy

Blood collection and processing: At each time point blood was collected in Lithium heparin (plasma) tubes and centrifuged at 12,000 rpm for 3 minutes. Plasma was stored at −80° C. prior to be analysis.

Necropsy: Upon sacrifice, brain and various tissues were dissected and rinsed in distilled water prior to homogenisation using an homogeniser and a known quantity of milliQ water and stored 80° C. prior analysis.

7.4.3 Sample Processing

7.4.3.1 Plasma Samples

Plasma samples were processed by addition of acetonitrile containing internal standard: Fifty (50) μL of plasma or urine samples were added to 200 μL of internal standard and vortexed for 10 seconds; spun for 3 minutes at 13400 rpm.

One hundred and fifty (150) μL of supernatant was transferred to a polypropylene vial. Two (2) μL of each processed sample was analysed by LCMSMS.

7.4.3.2 Organ Extraction

Upon sacrifice, brain and various tissues were dissected and homogenised individually in a glass/PTFE tissue homogeniser with the addition of water at a ratio of 0.5 mL/g for brain.

After homogenisation, the tissues were extracted with acetonitrile containing internal standard. Acetonitrile was added at a ratio of 1 part of water to 4 parts acetonitrile and centrifuged for 3 minutes at 13400 rpm. Two (2) μL of each processed tissue sample was analysed by LCMSMS.

8.0. Analytical Results

TABLE 12
CALIBRATION RESULTS - ANALYTICAL SET 1
Calibration Curve Source: QN1308 20161101 result

| | Idronoxil (TI/608) | | |
|---|---|---|---|
| | Expected | Calculated | |
| | Conc. | Conc. | |
| Standard | (ng/mL) | (ng/mL) | % Dev. |
| Solvent Blank | 0 | N/D | N/D |
| Internal Standard | 0 | N/D | N/D |
| C8 | 0.5 | N/D | N/D |
| C7 | 1 | 1.2 | 18.6 |
| C6 | 5 | 4.3 | −13.7 |
| C5 # | 10 | 8.6 | −13.7 |
| C4 | 50 | 47.7 | −4.6 |
| C3 | 100 | 98.5 | −1.5 |
| C2 | 500 | 507.6 | 1.5 |
| C1 | 1000 | 996.7 | −0.3 |

Notes:
N/D, not detected;
#, not used in calibration curve, correlation coefficient r² = 0.999

TABLE 14
CALIBRATION RESULTS - ANALYTICAL SET 2
Calibration Curve Source: QN1308 20161104 result

| | Idronoxil (TI/608) | | |
|---|---|---|---|
| | Expected | Calculated | |
| | Conc. | Conc. | |
| Standard | (ng/mL) | (ng/mL) | % Dev. |
| Solvent Blank | 0 | N/D | N/D |
| Matrix Blank | 0 | N/D | N/D |
| Internal Standard | 0 | N/D | N/D |
| C8 # | 0.5 | 0.9 | 74.5 |
| C7 | 1 | 1.2 | 15.8 |
| C6 | 5 | 4.3 | −13.3 |
| C5 | 10 | 10.2 | 2.1 |
| C4 | 50 | 46.4 | −7.2 |
| C3 | 100 | 102.0 | 2 |
| C2 | 500 | 503.5 | 0.7 |
| C1 | 1000 | 998.4 | −0.2 |

Notes:
N/D, not detected;
#, not used in calibration curve, correlation coefficient r² = 0.999

TABLE 15
QUALITY CONTROL SAMPLES RESULTS - ANALYTICAL SET 2
QC samples - Source: QN1308 20161104 result

| | Idronoxil (TI/608) | | |
|---|---|---|---|
| | Expected | Calculated | |
| Quality | Conc. | Conc. | |
| Control | (ng/mL) | (ng/mL) | % Dev. |
| QCL | 12 | 11.9 | −0.7 |
| QCL | 12 | 11.1 | −7.7 |
| QCL | 12 | 12.6 | 5.1 |
| Mean | — | 11.9 | — |
| SD | — | 0.77 | — |
| CV (%) | — | 6.5 | — |
| Accuracy (%) | — | 101.1 | — |
| QCM | 120 | 122.1 | 1.8 |
| QCM | 120 | 157.0 | 30.8 |
| QCM | 120 | 165.4 | 37.8 |
| Mean | — | 148.2 | — |
| SD | — | 22.94 | — |
| CV (%) | — | 15.5 | — |
| Accuracy (%) | — | 81.0 | — |
| QCH | 600 | 611.8 | 2 |
| QCH | 600 | 714.9 | 19.2 |
| QCH | 600 | 796.5 | 32.8 |
| Mean | — | 707.7 | — |
| SD | — | 92.59 | — |
| CV (%) | — | 13.1 | — |
| Accuracy (%) | — | 84.8 | — |

TABLE 19
Body weights

| | | Dose/Animal (mg) | | | | | Weight (g) | | |
|---|---|---|---|---|---|---|---|---|---|
| Animals | Sex | D1 | D2 | D3 | D4 | D5 | D1 | D2 | D6 |
| GROUP 1 | | | | | | | | | |
| No Treatment | | | | | | | | | |
| R5696 | M | — | — | — | — | — | 251 | — | — |
| R5697 | M | — | — | — | — | — | 239 | — | — |
| Mean | M | — | — | — | — | — | 245 | — | — |
| STDEV | | — | — | — | — | — | 8.5 | — | — |
| GROUP 2 | | | | | | | | | |
| TI/614 Acute | | | | | | | | | |
| R5698 | M | 50.6 | — | — | — | — | — | 253 | — |
| R5699 | M | 50.4 | — | — | — | — | — | 255 | — |
| R5700 | M | 50.9 | — | — | — | — | — | 263 | — |
| R5701 | M | 50.6 | — | — | — | — | — | 253 | — |
| Mean | M | 50.6 | — | — | — | — | — | 256 | — |
| STDEV | | 0.21 | — | — | — | — | — | 4.8 | — |
| GROUP 3 | | | | | | | | | |
| TI/614 Repeated | | | | | | | | | |
| R5702 | M | 50.1 | 50.2 | 50.6 | 50.7 | 50.8 | — | — | 312 |
| R5703 | M | 50.6 | 50.8 | 50.5 | 50.3 | 50.9 | — | — | 328 |
| R5704 | M | 50.2 | 50.7 | 50.9 | 50.2 | 50.7 | — | — | 321 |
| R5705 | M | 50.6 | 50.3 | 50.7 | 50.2 | 50.8 | — | — | 313 |
| R5706 | M | 50.8 | 50.2 | 50.7 | 50.9 | 50.5 | — | — | 318 |
| Mean | M | 50.5 | 50.4 | 50.7 | 50.8 | 50.7 | — | — | 318 |
| STDEV | | 0.30 | 0.29 | 0.15 | 0.32 | 0.15 | — | — | 6.5 |

TABLE 20A
Organ Weights Brain Extraction

| | | [symbol] | Total | Total | Organ | [symbol] |
|---|---|---|---|---|---|---|
| Animals | Sex | Detected Conc. | Extraction Volume | Amount | Weight | Detected Conc. |
| Brain Extraction | | (ng/mL) | (mL) | (ng) | (g) | (ng/g) |
| GROUP 1 | | | | | | |
| No Treatment | | | | | | |
| R5696 | M | 0.0 | 4.545 | 0.0 | 1.9377 | 0.0 |
| R5697 | M | 0.0 | 4.580 | 0.0 | 1.8657 | 0.0 |
| Mean | M | 0.0 | 4.781 | 0.0 | 1.5017 | 0.0 |
| STDEV | | 0.00 | 0.1398 | 0.00 | 0.06891 | 0.00 |
| GROUP 2 | | | | | | |
| TI/614 Acute | | | | | | |
| R5698 | M | 2.4 | 4.000 | 21.3 | 1.8410 | 6.1 |
| R5699 | M | 4.0 | 4.235 | 18.9 | 1.8934 | 10.0 |
| R5700 | M | 0.7 | 4.545 | 44.1 | 1.8175 | 24.3 |
| R5701 | M | 3.6 | 4.390 | 16.4 | 1.8169 | 8.9 |
| Mean | M | 4.9 | 4.618 | 22.7 | 1.8472 | 12.3 |
| STDEV | | 3.25 | 0.0819 | 14.63 | 0.03246 | 8.12 |
| GROUP 3 | | | | | | |
| TI/614 Repeated | | | | | | |
| R5702 | M | 0.8 | 4.785 | 11.2 | 1.9188 | 6.9 |
| R5703 | M | 7.8 | 4.915 | 38.7 | 1.9896 | 10.4 |
| R5704 | M | 2.5 | 4.765 | 12.2 | 1.9038 | 6.3 |
| R5705 | M | 2.8 | 4.530 | 12.2 | 1.8126 | 7.3 |
| R5706 | M | 2.5 | 5.008 | 12.3 | 2.0012 | 6.1 |
| Mean | M | 3.7 | 4.811 | 17.9 | 1.9246 | 9.2 |
| STDEV | | 2.29 | 6.1927 | 11.63 | 0.67664 | 8.73 |

[symbol]

indicates data missing or illegible when filed

TABLE 21
Plasma Concentration

| | | Idronoxil Detected Concentration (ng/mL) | | | | | |
|---|---|---|---|---|---|---|---|
| | | Day 1 | | | Day 5 | | |
| Animals | Sex | 30 Minutes | 2 Hours | 24 Hours | 30 Minutes | 2 Hours | 24 Hours |
| GROUP 1 | | | | | | | |
| No Treatment | | | | | | | |
| R5696 | M | N/D | — | — | — | — | — |
| R5697 | M | 0.886 | — | — | — | — | — |
| Mean | M | 0.886 | — | — | — | — | — |
| STDEV | | — | — | — | — | — | — |
| GROUP 2 | | | | | | | |
| TI/614 Acute | | | | | | | |
| R5698 | M | 57.857 | 27.915 | 0.757 | — | — | — |
| R5699 | M | 110.010 | 11.151 | N/D | — | — | — |
| R5700 | M | — | 4.560 | 6.761 | — | — | — |
| R5701 | M | — | 34.781 | N/D | — | — | — |
| Mean | M | 86.937 | 19.677 | 3.759 | — | — | — |
| STDEV | | 41.1246 | 14.0028 | 4.2455 | — | — | — |
| GROUP 3 | | | | | | | |
| TI/614 Repeated | | | | | | | |
| R5702 | M | 11.188 | 22.005 | 0.862 | 14.171 | 9.649 | N/D |
| R5703 | M | 24.001 | 8.873 | 0.853 | 28.615 | 10.506 | N/D |
| R5704 | M | 63.565 | 8.475 | 0.735 | 27.046 | 5.061 | N/D |
| R5705 | M | 4.195 | 1.998 | 1.278 | 11.088 | 26.018 | N/D |
| R5706 | M | 138.468 | 23.411 | 1.780 | 32.500 | 24.008 | N/D |
| Mean | M | 48.283 | 12.864 | 1.102 | 22.700 | 15.208 | — |
| STDEV | | 55.3976 | 9.4580 | 0.4313 | 9.4746 | 9.5518 | — |

Note:
“N/D”, Not Detected.

### REFERENCES

Lambert et al, 2005; Williamson and Manach, 2005
Piao M, Mori D, Satoh T, et al. (2006). Inhibition of endothelial cell proliferation, in vitro angiogenesis, and the down-regulation of cell adhesion-related genes by genistein. Combined with a cDNA microarray analysis. Endothelium 13(4):249-66
Adjakly M, Ngollo M, Dagdemir A, Judes G, Pajon A, Karsli-Ceppioglu S, Penault-Llorca F, Boiteux J P, Bignon Y J, Guy L, Bernard-Gallon D. (2015). Prostate cancer: the main risk and protective factors—epigenetic modifications. Ann Endoncrinol (Paris) 76:25-41.
Kazi A, Daniel K G, Smith D M, et al. (2003). Inhibition of the proteasome activity, a novel mechanism associated with the tumor cell apoptosis-inducing ability of genistein. Biochem Pharmacol 66(6):965-76.
Guy L, Vedrine N, Urpi-Sarda M, Gil-lzguierdo A, Al-Maharik N, Boiteaux J P, Scalbert A, Remesy C, Botting N P, Manach C. (2008). Orally administered isoflavones are present as glucosides in the human prostate. J Nutr 60:461-8.
Zhang Y, Hendrich S, Murphy P A. (2003). Glucuronides are the main isoflavone metabolites in women. J Nutr 133:399-404.
King C D, Rios G R, Green M D, Tephly T R (2000). UDP-glucuronosyltransferases. Curr Drug Metab 1:143-161.
Guillemette C. (2003). Pharmacogenomics of human UDP-glucuronosyltransferase enzymes. Pharmacogenomics J. 3:136-158.
Maruo Y, Iwai M, Mori A, Sato H, Takeuchi Y. (2005). Polymorphism of UDP-glucuronosyltransferase and drug metabolism. Curr Drug Metab 6:91-99.
Wu B, Kulkarni K, Basu S, Zhang S, Hu M. (2011). First-pass metabolism via UDP-glucuronosyltransferase: a barrier to oral bioavailability of phenolics. J Pharmaceutical Sci 100:3655-3681.
Howes J B, de Souza P L, West L, Huang L J, Howes L G. Pharmacokinetics of phenoxodiol, a novel isoflavone, following intravenous administration to patients with advanced cancer. BMC Clin Pharmacol. 2011 Feb. 3; 11:1.
Brown D, Heaton A, Husband A (2008). Idronoxil. Drugs Fut. 33(10):844-860.
Westholm D E et al. The blood-brain barrier thyroxine transporter organic anion-transporting polypeptide 1c1 displays atypical transport kinetics. Endocrinology 150, 5153-5162, 2009.
Youdim K et al. Flavonoid permeability across an in situ model of the blood-brain barrier. Free Rad Biol Med 36, 592-604, 2004)
Tsai T H. Concurrent measurement of unbound genistein in the blood, brain and bile of anaesthetized rats using microdialysis and it pharmacokinetic application. J Chromatogr A 1073:317-322, 2005).

1. A method for providing a compound of general formula (I) 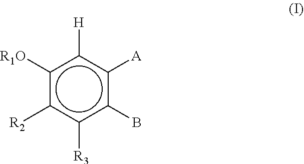
wherein
R₁ is H, C₁₋₁₀ alkyl, aryl, arylalkyl or R_{A}CO where R_{A} is C₁₋₁₀ alkyl or an amino acid;
R₂ is H, OH or OR₆;
R₃ is H, OH, or R_{B} where R_{B} is an amino acid or COR_{A} where R_{A} is as previously defined;
A and B together with the atoms between them form a six membered ring selected from the group 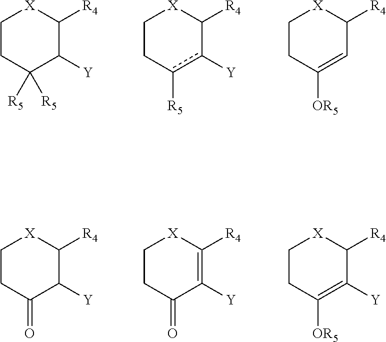
or A is —OR₆ and B is: 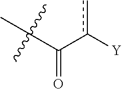
wherein
R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where R_{C} is as previously defined, or CONHR_{E} where R_{E} is as previously defined;
R₅ is H, CO₂R_{C} where R_{C} is as previously defined, or COR_{C}OR_{E} where R_{C} and R_{E} are as previously defined, and where the two R₅ groups are attached to the same group they are the same or different;
R₆ is C₁₋₁₀ alkyl, aryl or arylalkyl;
X is O, N or S;
Y is 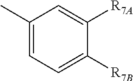
where R_{7A} and R_{7B} are each independently H, OH or OR₇; with the proviso that at least one of R_{7A} and R_{7B} is OH or OR₇;
wherein R₇ is C₁₋₁₀ alkyl, aryl or arylalkyl; and
“ [symbol] ” represents either a single bond or a double bond;
to the central nervous system (CNS), especially to brain tissue, of an individual including the step of rectally administering the compound of general formula (I) to an individual requiring provision of the compound to the CNS, thereby providing the compound to the CNS of the individual.

2. (canceled)

3. The method of claim 1, wherein the compound is selected from the group consisting of 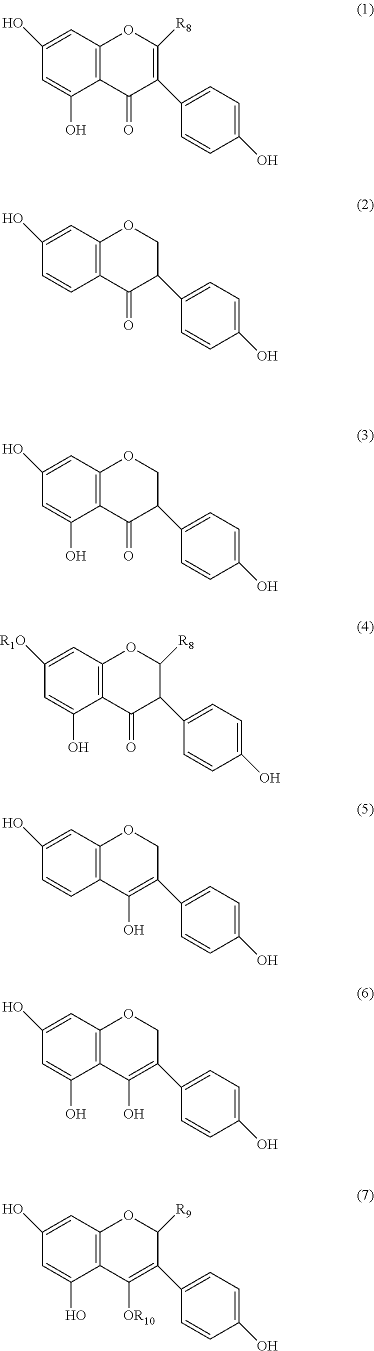 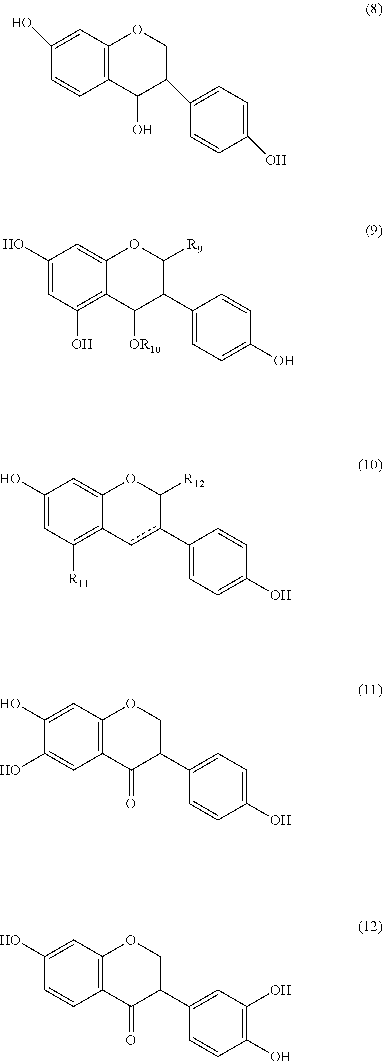
wherein
R₈ is H or COR_{D} where R_{D} is as previously defined;
R₉ is CO₂R_{C} or COR_{E} where R_{C} and R_{E} are as previously defined;
R₁₀ is COR_{C} or COR_{C}OR_{E} where R_{C} and R_{E} are as previously defined;
R₁₁ is H or OH;
R₁₂ is H, COOH, CO₂R_{C} where R_{C} is as previously defined, or CONHR_{E} where R_{E} is as previously defined; and
“ [symbol] ” represents either a single bond or a double bond.

4. The method of claim 1, wherein the compound is selected from:

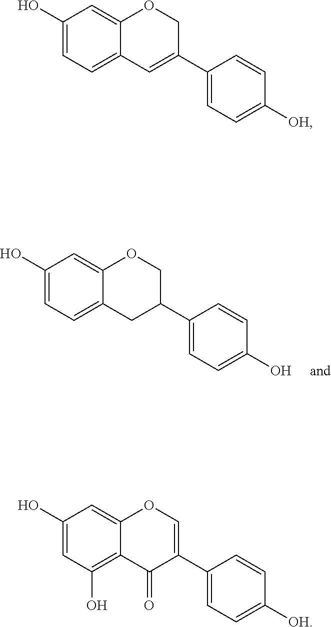 [SMILES: Oc1ccc(C2=Cc3ccc(O)cc3OC2)cc1 ; Oc1ccc(C2COc3cc(O)ccc3C2)cc1 ; "and" ; O=c1c(-c2ccc(O)cc2)coc2cc(O)cc(O)c12]

5. The method of claim 4, wherein the compound is

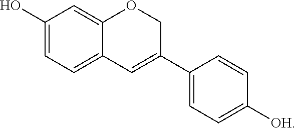 [SMILES: Oc1ccc(C2=Cc3ccc(O)cc3OC2)cc1]

6-9. (canceled)

10. A method for treating an individual for brain cancer including the step of rectally, vaginally or through urethral application, administering a formulation including a lipophilic base and a compound of general formula (I) to the individual.

11. The method of claim 10 wherein the brain cancer is glioblastoma multiforme.

12. The method of claim 11 wherein the compound is:

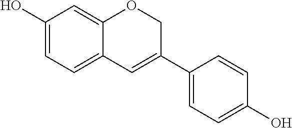 [SMILES: Oc1ccc(C2=Cc3ccc(O)cc3OC2)cc1]

13. The method of claim 10 wherein the individual has received, or is receiving temozolomide for treatment of the brain cancer.

14. The method of claim 10 wherein the compound of general formula (I) is provided to the individual to sensitise the cancer cells of the individual to the cytotoxic effects of temozolomide.
